(19) United States
(12) Patent Application Publication
(10) Pub. No.: US 2023/0127148 A1
(43) Pub. Date: Apr. 27, 2023

(54) TAMPER RESISTANT DEVICES

(71) Applicants: KEVIN B. WILLIAMS, CLEARWATER, FL (US); MARYA L. SEARCY, TAMPA, FL (US)

(72) Inventors: KEVIN B. WILLIAMS, CLEARWATER, FL (US); MARYA L. SEARCY, TAMPA, FL (US)

(21) Appl. No.: 17/964,469

(22) Filed: Oct. 12, 2022

Related U.S. Application Data
(60) Provisional application No. 63/270,970, filed on Oct. 22, 2021.

(51) Int. Cl. A61M 5/50; A61M 39/20

(52) U.S. Cl. CPC A61M 5/5086; A61M 39/20; A61M 2039/0285

(57) ABSTRACT

The present invention is directed to tamper resistant devices for discouraging and/or preventing unauthorized persons, such as patients, from tampering with access to in-situ intravenous (IV) medical catheter devices (placed either centrally or peripherally) and their corresponding medical fluid lines with access ports. The tamper resistant devices are particularly designed to prevent unauthorized patient access to the IV and to prevent any insertion/injection of non-prescribed medications/foreign substances through their IV access and associated ports.

### RELATED APPLICATION

This application claims the benefit of U.S. Provisional Pat. Application No. 63/270,970, filed on Oct. 22, 2021.

### TECHNICAL FIELD

The present invention relates generally to tamper resistant devices and more specifically relates to tamper resistant enclosures that are designed to prevent unauthorized access to an IV catheter, its ports or any open ports on medical fluid lines.

### BACKGROUND INFORMATION

There are numerous circumstances in which a patient may require the placement of an intravenous (IV) catheter. A few examples include: the administration of IV fluids for hydration, electrolyte replacement, antibiotic therapy, administration of injectable narcotics (i.e., patient-controlled analgesia/PCA), blood administration and/or sampling (physician ordered lab work), and dialysis therapies. Patients may have these central or peripherally placed IV catheters in place for varying lengths of time; from a few hours to several months. One imperative is that the IV catheters and medical fluid lines access ports (aka Leurs, Claves) are only accessed by trained medical professionals after being appropriately disinfected to prevent the introduction of bacterial agents into the patient’s bloodstream.

The prevention and detection of patient tampering with these injectable access ports is critically important in both the inpatient and outpatient settings. Patients with IV catheters possess a multitude of physical and mental conditions that can contribute to an individual’s intent to either intentionally or unintentionally tamper with their IV catheter or access ports by disconnecting prescribed medical treatments or by self-injecting substances not medically prescribed into them. All these actions lead to increased hospital lengths of stay for inpatients, extended outpatient treatments, delayed healing, potential for infection, potential transmission of blood borne pathogens, prolongation of opiate abuse/addiction, potential overdose and death.

Hospitals have a duty to protect patients from harm. Currently, there are no effective safety devices that exist to prevent patients from accessing and tampering with indwelling IV catheter access ports and/or the access ports on any medical fluid lines. The Centers for Medicare and Medicaid Services (CMS) have identified 8 high-cost, high-frequency events they do not reimburse for and consider “never-events”. One of these never-events is hospital acquired bloodstream infections. No effective devices exist which are capable of ensuring that IV catheters and medical fluid line access ports are only accessed by trained medical professionals and of reducing the risks associated with a blood stream infection.

Consequently, there is a need for a tamper resistant device that is effective for the prevention and detection of tampering with medical fluid lines access ports by unauthorized persons.

### SUMMARY

The present invention is directed in the creation of tamper resistant devices that can effectively discourage and/or prevent both hospitalized and home health patients from tampering with access to in-situ intravenous (IV) medical catheter devices (placed either centrally or peripherally) and their corresponding medical fluid lines with access ports. The tamper resistance devices of the present invention are particularly effective to prevent disconnection of such medical fluid lines and to provide evidence of either tampering or disconnection.

The devices of the present invention are aimed at ensuring that IV catheters and medical fluid line access ports are only accessed by trained medical professionals and at reducing the risks associated with a blood stream infection.

In a first embodiment, the tamper resistant device comprises first and second shell members each having an interior space and connecting means for removably connecting the first and second shell members together. The second shell member has an outer diameter portion smaller than an inner diameter of the first shell member so that in a connected state, the second shell member is received by the interior space of the first shell member and an inner circumferential surface portion of the first shell member encircles and covers an outer circumferential surface portion of the second shell member. In the connected state, the first and second shell members together form an interior space for holding a portion of an access port of a fluid line to prevent tampering with the access port by unauthorized persons.

As an exemplary feature of the first embodiment, the means for removably connecting the first and second shell members together comprises at least one locking element formed in the interior space of the first shell member and at least one locking hole formed in the second shell member for removably receiving the at least one locking element.

As other exemplary features of the first embodiment, each of the first shell member and the second shell member has a unitary structure made of a single piece of material, and/or made of a flexible material, and/or having a generally semi-circular cross-section.

As yet another exemplary feature, the tamper resistant device of the first embodiment further comprises tamper detection means for providing a visual indication of tampering with the first and/or second shell members. As an example, the tamper detection means comprises one of a tamper detection sticker and a tamper evident shrink band or wrap applied to a preselected region of the first and/or second shell members.

In a second embodiment, the tamper resistant device comprises a shell member having a unitary structure made of a single piece of material, the shell member having a first tubular portion with a first interior space and a second tubular portion with a second interior space and extending from the first tubular portion, the first and second interior spaces being configured to receive and retain therein an access port of a fluid line to prevent tampering with the access port by unauthorized persons. The tamper resistant device of the second embodiment further comprises means for removably holding and retaining the access port within the first and second interior spaces of the first and second tubular portions of the shell member.

As one exemplary feature of the second embodiment, the means for removably holding and retaining the access port comprises a locking element separate from the shell member and a through-hole formed in the first tubular portion of the shell member for removably receiving the locking element so as to engage the access port retained in the first interior space of the first tubular portion.

As another exemplary feature of the second embodiment, the tamper resistant device further comprises a tool member for removing the locking element from the through-hole of the first tubular portion to permit removal of the access port from the first and second tubular portions.

As yet another exemplary feature, the tamper resistant device of the second embodiment further comprises tamper detection means for providing a visual indication of tampering with the shell member. As an example, the tamper detection means comprises one of a tamper detection sticker and a tamper evident shrink band or wrap applied to a preselected region of the shell member.

In a third embodiment, the tamper resistant device comprises a cap member having an open end providing access into an interior space of the cap member configured to receive and removably retain therein an open port of a fluid line, and means for removably holding and retaining the open port within the interior space of the cap member to prevent unauthorized tampering with the open port by unauthorized persons.

As one exemplary feature of the third embodiment, the means for removably holding and retaining the open port comprises a locking element and a through-hole formed in the cap member for removably receiving the locking element so that the locking element engages and retains the access port within the interior space of the cap member.

As another exemplary feature of the third embodiment, the tamper resistant device further comprises a disinfecting element disposed within the interior space of the cap member for contacting and maintaining in a disinfected state the access port disposed in the interior space.

As yet another exemplary feature of the third embodiment, the tamper resistant device further comprises detection means for providing a visual indication of tampering with the cap member. As an example, the tamper detection means comprises one of a tamper detection sticker and a tamper evident shrink band or wrap applied to a preselected region of the cap member.

These and other features and advantages of the present invention will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is an elevated perspective view of a tamper resistance device according to a first embodiment of the present invention, showing the two (first and second) shell members of the device in a connected state.

FIG. 2 is an elevated perspective view of the tamper resistant device of the first embodiment, showing the two shell members in a disconnected state.

FIG. 3 is a bottom view of the tamper resistant device of the first embodiment, showing the two shell members in the disconnected state.

FIG. 4 is a bottom view of the tamper resistant device of the first embodiment, showing the two shell members in the connected state.

FIG. 5 is a top view of the tamper resistant device of the first embodiment, showing the two shell members in the connected state.

FIG. 6 is a front elevational view of the tamper resistant device of the first embodiment, showing the two shell members in the connected state.

FIG. 7 is a side elevational view of the tamper resistant device of the first embodiment, showing the two shell members in the connected state.

FIG. 8 is a front elevational view of the first shell member of the tamper resistant device according to the first embodiment.

FIG. 9 is a front elevational view of the second shell member of the tamper resistant device according to the first embodiment.

FIG. 10 is an elevational perspective view of the tamper resistant device of the first embodiment, showing the second shell member engaging an access port of a medical fluid line and the first shell member disconnected from the second shell member.

FIG. 11 is another elevational perspective view of the tamper resistant device of the first embodiment, showing the second shell member engaging an access port of a medical fluid line and the first shell member disconnected from the second shell member.

FIG. 12 is an elevational side view of the tamper resistant device of the first embodiment engaging an access port of a medical fluid line, with the first and second shell members being connected to one another as shown in FIGS. 1, 4, 5, 6 and 7.

FIG. 13 is a cross-sectional view taken along line A-A in FIG. 12.

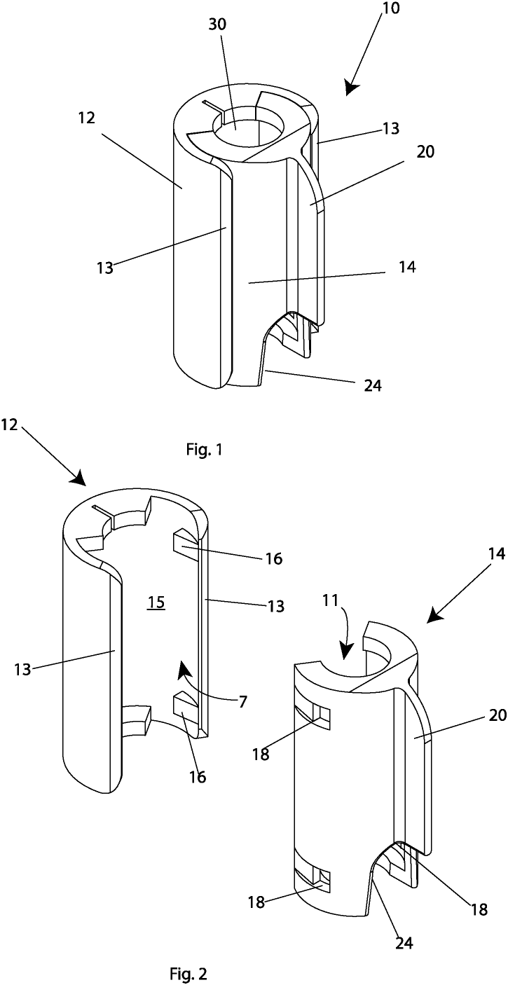

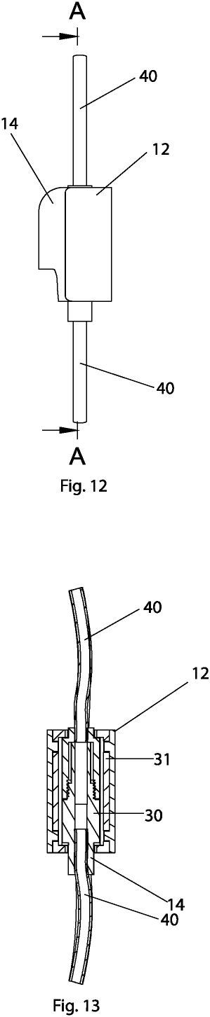

FIG. 12 is a rear elevational view of the tamper resistance device of FIG. 1 engaging an access port of a medical fluid line.

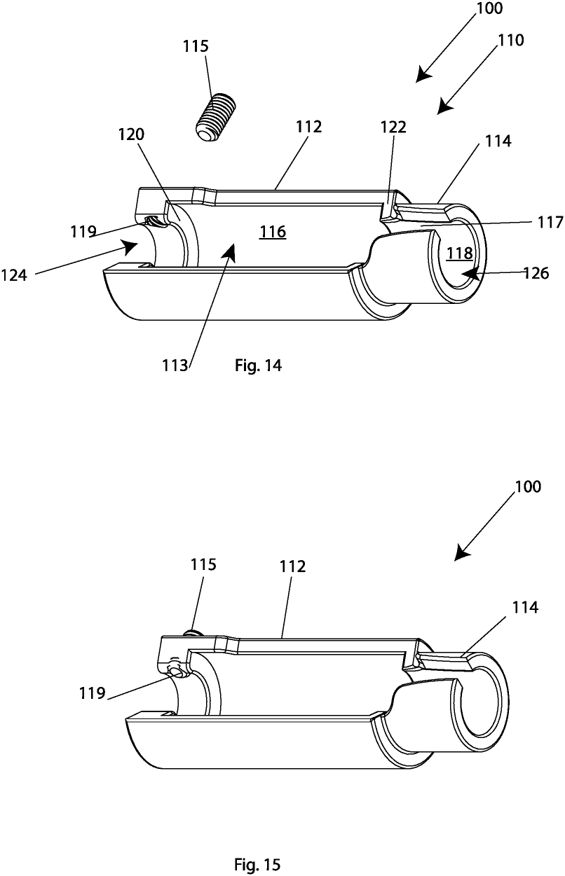

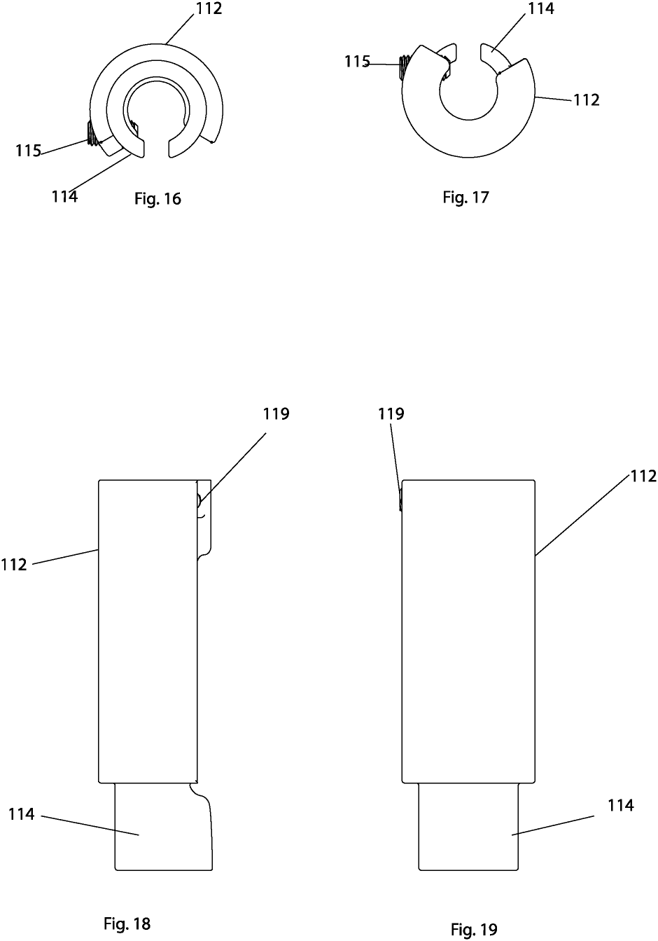

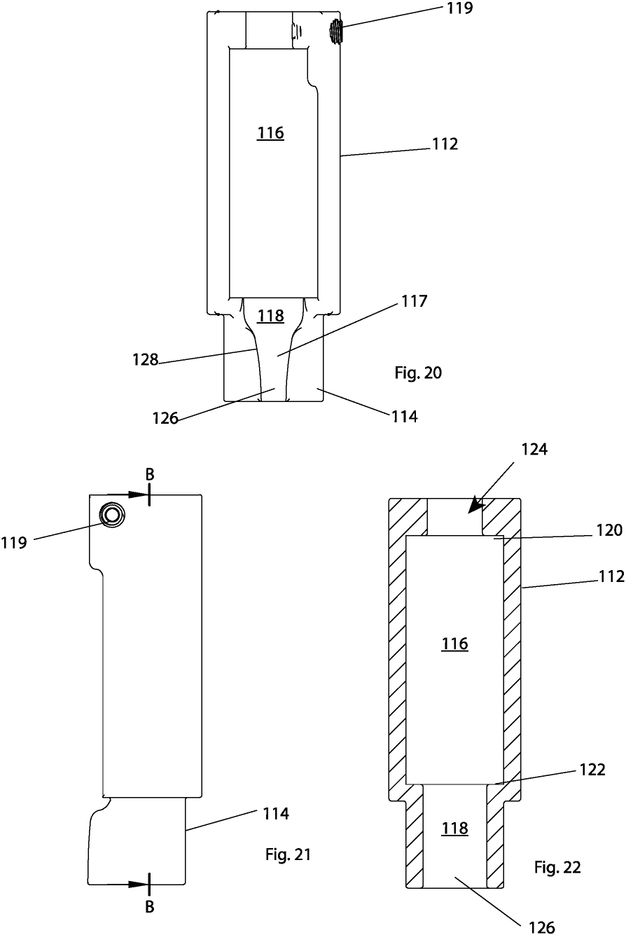

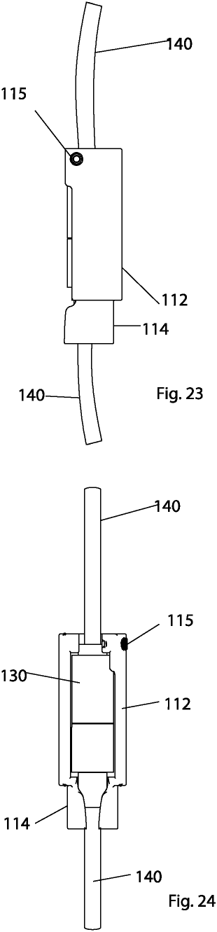

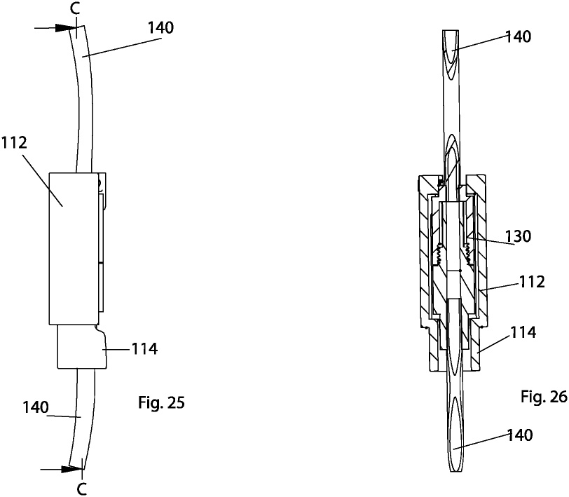

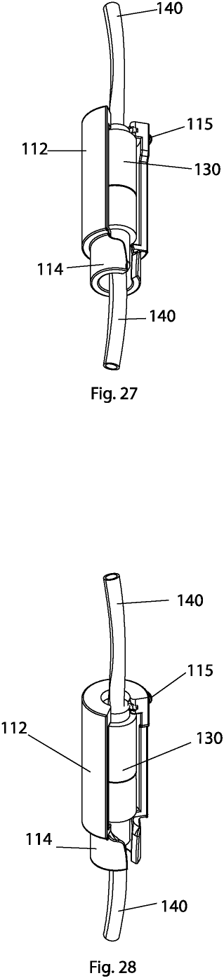

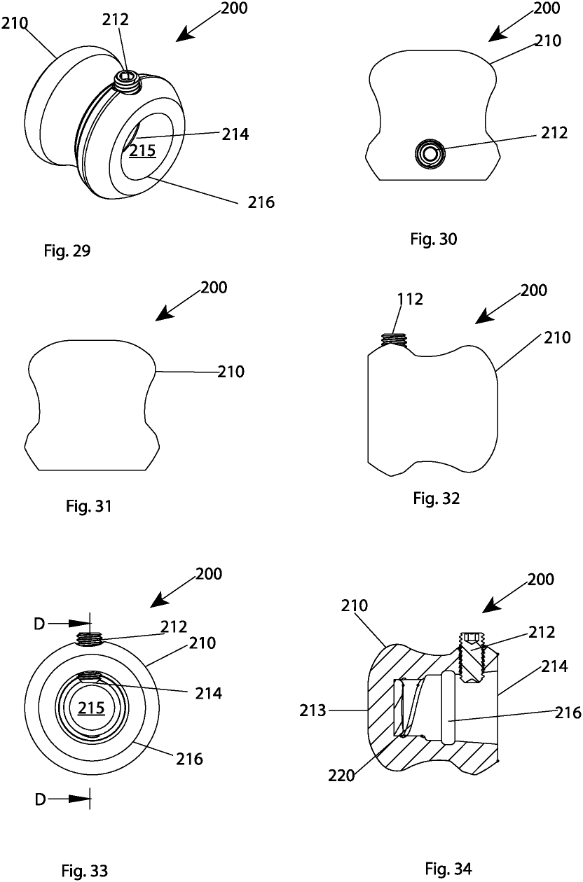

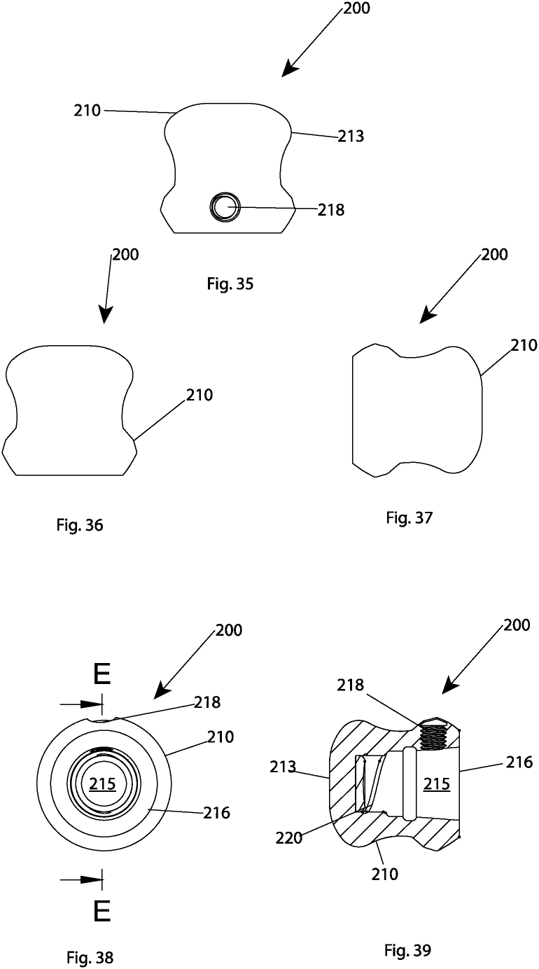

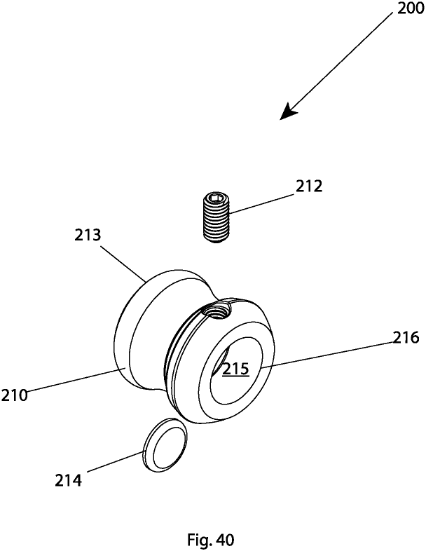

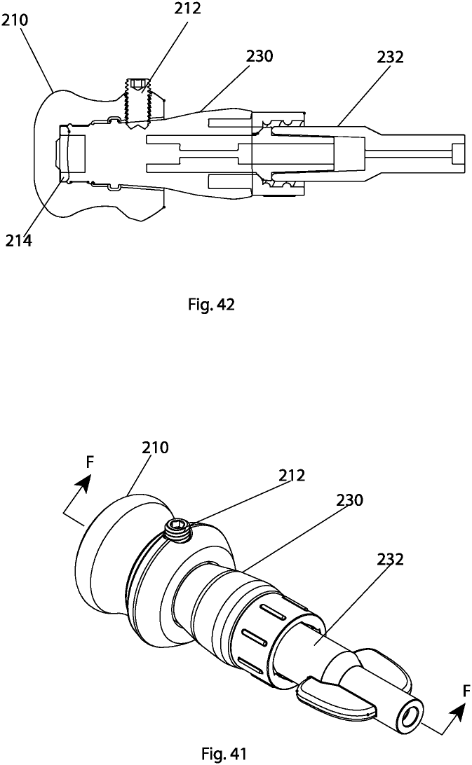

FIG. 13 is a cross-sectional view taken along line B-B in FIG. 12.

FIG. 14 is an exploded perspective view of a tamper resistant device according to a second embodiment of the present invention, showing a locking element in a removed state.

FIG. 15 is a view like FIG. 14 except showing the locking element in an assembled state.

FIG. 16 is a top view of the tamper resistant device of the second embodiment.

FIG. 17 is a bottom view of the tamper resistant device of the second embodiment.

FIG. 18 is a left side view of the tamper resistant device of the second embodiment.

FIG. 19 is a rear view of the tamper resistant device of the second embodiment.

FIG. 20 is a front view of the tamper resistant device of the second embodiment.

FIG. 21 is a right-side view of the tamper resistant device of the second embodiment.

FIG. 22 is a cross-sectional view taken along line B-B in FIG. 21.

FIG. 23 is a right-side view of the tamper resistant device of the second embodiment in an assembled state engaging an access port of a medical fluid line.

FIG. 24 is a front view of the tamper resistant device of the second embodiment in an assembled state engaging an access port of a medical fluid line.

FIG. 25 is a left side view of the tamper resistant device of the second embodiment in an assembled state engaging an access port of a medical fluid line.

FIG. 26 is a cross-sectional view taken along line C-C in FIG. 25.

FIG. 27 is a perspective view of the tamper resistant device of the second embodiment in an assembled state engaging an access port of a medical fluid line.

FIG. 28 is another perspective view of the tamper resistant device of the second embodiment in an assembled state engaging an access port of a medical fluid line.

FIG. 29 is a perspective view of a tamper resistant device according to a third embodiment of the present invention.

FIG. 30 is a front elevational view of the tamper resistant device of the third embodiment.

FIG. 31 is a rear elevational view of the tamper resistant device of the third embodiment.

FIG. 32 is a side view of the tamper resistant device of the third embodiment.

FIG. 33 is an end view of the tamper resistant device of the third embodiment.

FIG. 34 is a cross-sectional view taken along the line D-D in FIG. 33.

FIGS. 35-39 are views similar to FIGS. 30-34, respectively, except that a locking element of the tamper resistant device is removed, where FIG. 35 is a front elevational view, FIG. 36 is a rear elevational view, FIG. 37 is a side view, FIG. 38 is an end view, and FIG. 39 is a cross-sectional view taken along the line E-E in FIG. 38.

FIG. 40 is an exploded view of the tamper resistant device of the third embodiment.

FIG. 41 is a perspective view of the tamper resistant device of the third embodiment in an assembled state engaging an access port of a medical fluid line.

FIG. 42 is a cross-sectional view taken along line F-F in FIG. 41.

### DETAILED DESCRIPTION OF PREFERRED EMBODIMENTS

The foregoing summary, as well as the following detailed description of preferred embodiments of the disclosure, will be better understood when read in conjunction with the accompanying drawings. For the purpose of illustrating the various aspects of the tamper resistant devices of the present disclosure, there is shown in the drawings embodiments which are presently preferred. It should be understood, however, that the various aspects of the tamper resistant devices of the present disclosure are not limited to the precise arrangement and instrumentalities shown.

The word “exemplary” or “example” is used herein to mean “serving as an example, instance, or illustration.” Any embodiment described herein as “exemplary” or “example” is not necessarily to be construed as preferred or advantageous over other embodiments. The images in the drawings are simplified for illustrative purposes and are not depicted to scale. To facilitate understanding, identical reference numerals have been used, where possible, to designate identical elements that are common to the figures, except that suffixes may be added, when appropriate, to differentiate such elements.

### Introduction

The misuse of and addiction to opiates is a national public health crisis. The Centers for Disease Control and Prevention (CDC) estimate that 50,000(+) people die yearly from opiate overdoses with a yearly economic burden greater than 78 billion dollars for healthcare costs, addiction treatment, lost productivity, and criminal justice proceedings.

In the 1990’s pharmaceutical companies assured the medical community that patients would not become addicted to prescription opioids. This caused medical providers to prescribe opiates at increased rates and led to wide-spread diversion and misuse/abuse of these medications. The National Institutes of Health (NIH) has partnered with the pharmaceutical companies and academic research centers to develop non-addictive pain management strategies, innovative medications and technologies to treat opioid use disorders, and strategies/technologies to prevent or reverse the misuse/abuse of opioid medications.

The various embodiments of the tamper resistant devices according to the present invention have been developed to prevent hospitalized and home health patients from tampering with access to in-situ intravenous (IV) medical catheter devices (placed either centrally or peripherally) and their corresponding medical fluid lines with access ports.

There are numerous circumstances in which a patient may require the placement of an intravenous (IV) catheter. A few examples include: the administration of IV fluids for hydration, electrolyte replacement, antibiotic therapy, administration of injectable narcotics (i.e. patient controlled analgesia/PCA), blood administration and/or sampling (physician ordered lab work), and dialysis therapies. Patients may have these central or peripherally placed IV catheters in place for varying lengths of time; from a few hours to several months. One imperative is that the IV catheters and medical fluid lines access ports (aka Leurs, Claves) are only accessed by trained medical professionals after being appropriately disinfected to prevent the introduction of bacterial agents into the patient’s bloodstream. The tamper resistant devices according to the present invention have been designed to prevent unauthorized patient access to the IV and to prevent any insertion/injecting non prescribed medications/foreign substances through their IV access and associated ports.

The prevention and detection of patient tampering with these injectable access ports is critically important in both the inpatient and outpatient settings. By securing and preventing the tampering of the IV and any access points adds additional depth to patient safety. The tamper resistant devices of the present invention will help to minimize any potential blood stream infections risk, aid in detecting/identifying any potential abuse/self-injection of narcotic/opioid medications, and aid in the prevention of sharing and/or selling of continuous or patient on-demand intravenously administered opioid medications with other patients (this practice is often referred to as “bumping”).

Patients with IV catheters possess a multitude of physical and mental conditions that can contribute to an individual’s intent to either intentionally or unintentionally tamper with their IV catheter or access ports by disconnecting prescribed medical treatments or by self-injecting substances not medically prescribed into them. These actions all lead to increased hospital lengths of stay for inpatients, extended outpatient treatments, delayed healing, potential for infection, potential transmission of blood borne pathogens, prolongation of opiate abuse/addiction, potential overdose and death. The tamper resistant devices of the present invention possess both visual and escalating physical deterrent properties for IV access ports, where no current visual or physical barrier currently exists in the inpatient or outpatient setting to prevent tampering.

The embodiments of the tamper resistant devices according to the present invention are described below with reference to the drawings.

### First Embodiment

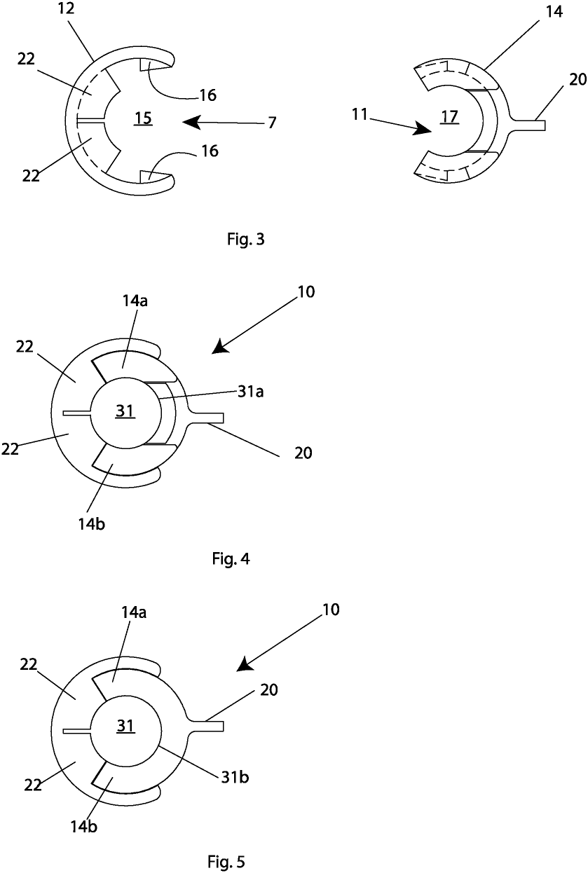

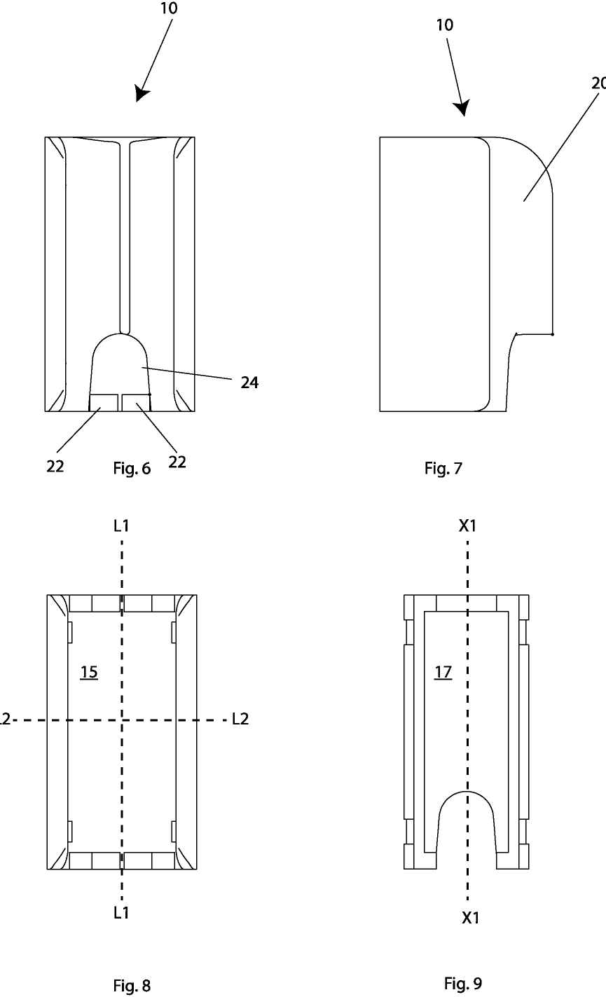

FIGS. 1-13 illustrate a first embodiment of the tamper resistant device, generally designated at 10, according to the present invention.

Tamper resistant device 10 has a first shell member 12 having a generally semi-annular cross-section and a second shell member 14 having a generally semi-annular cross-section. Each of first and second shell members 12, 14 have a generally tubular construction and are configured for engagement with and placement over an access port of a medical fluid line as further described below.

First shell member 12 has an open end 7 providing access into an interior space 15 arranged with two opposing sets (four in total) of locking tabs 16 directed outwardly from an interior surface of first shell member 12. Second shell member 14 has an open end 11 providing access into an interior 17 provided with two opposing sets (four in total) of locking holes 18 (through-holes) configured for removably receiving respective locking tabs 16 of first shell member 12 to place first and second shell members 12, 14 in an assembled or locked state.

Second shell member 14 is also provided with a holding portion 20 to facilitate its connection to and disconnection from first shell member 12 as further described below. In the locked state of tamper resistant device shown in FIG. 1, first and second shell members 12, 14 form an interior space 31 with opposite open ends 31a, 31b for accommodating an access port and corresponding medical fluid line, as further described below. Second shell member 14 also has a cut-away portion 24 adjacent one end of holding portion 20 for accommodating, together with interior space 31 and opposite open ends 31a, 31b, an access port and corresponding medical fluid line, as further described below. Locking tabs 16 of first shell member 12 and locking holes 18 of second shell member 12 define means for removably connecting first and second shell members 12, 14 together.

Although tamper resistance device 10 according to this embodiment is provided with four locking tabs 16 and four locking holes 18, it will be appreciated that a modified form of tamper resistance device 10 can be provided with less than or more than four locking tabs and corresponding locking holes, provided that such locking tab/hole combination sufficiently removably connects first and second shell members 112, 114 together to accomplish the objectives of the present invention.

FIGS. 1 and 2 show tamper resistant device 10 in assembled/locked and disassembled/unlocked states, respectively. Without considering holding portion 20, an outer diameter of second shell member 14 is configured to be sufficiently smaller than an internal diameter than the inner first shell member 12 such that in the locked state, first shell member 12 securely receives a circumferential surface portion of second shell member 14, as shown in FIG. 1. The material selected for first and second shell members 12, 14 has a degree of flexibility such that the first and second shell members 12, 14 can be locked together (FIG. 1) and disconnected from one another (FIG. 2) with only manual force and without requiring any tools, as further described below.

From the disassembled (disconnected) state shown in FIG. 2, first and second shell members 12, 14 can be assembled (connected) together by manually inserting second shell member 14 (of smaller diameter) into interior space 15 of first shell member 12 and manually pressing both together until locking tabs 16 of first shell member 12 engage locking holes 18 of second shell member 12 to thereby achieve the connected state shown in FIG. 1. When first and second shell members 12, 14 are pressed together as noted above, side portions 13 of first shell member 12 are slightly flexed outwardly to allow second shell member 14 to be further inserted (pressed) into interior space 15 of first shell member 12 until locking tabs 16 engage locking holes 18. The proper engagement between locking tabs 16 and locking holes 18 can be confirmed by a corresponding audible clicking sound.

In the locked state of tamper resistant device 10 shown in FIG. 1, interior surface portions of first shell member 12 extending from corresponding sides portions 13 encircle and cover exterior surface portions of second shell member 14 so that side portions 13 of first shell member 14 are spaced part from holding portion 20 of second shell member 14. In one embodiment, side portions 13 of first shell member 12 are equidistantly spaced from and on opposite sides of holding portion 20 of second shell member 14, as shown in FIGS. 4 and 5, for example.

From the assembled state shown in FIG. 1, disconnection of first and second shell members 12, 14 from one another can be accomplished by manually flexing outwardly one of the side portions 13 of first shell member 12 with one hand while slightly pulling second shell member 14 away from first shell member 12 with the other hand until one of the locking tabs 16 disengages from the corresponding locking hole 18, and then continuing pulling the first and second shell members 12, 14 away from each other to disengage the other locking tab 16 from the corresponding locking hole 18. Holding portion 20 of second shell member 14 facilitates holding and manipulation of second shell member 14 during the connection and disconnection processes described above.

First shell member 12 is provided with boss elements 22 extending into interior space 15 from opposing ends thereof, as shown in FIGS. 1-4, 8. Boss elements 22 are configured to contact respective end portions 14a, 14b of second shell member 14 in the connected state of first and second shell members 12, 14, as shown in FIGS. 1, 4 and 5. The circumferential extent of boss elements 22 is such that they are brought into contact with respective end portions 14a, 14b of second shell member 14 when locking tabs 16 of first shell member 12 are removably received by respective locking holes 18 of second shell member 14. At this point, the secure, yet removable connection between first and second shell members 12, 14 can be confirmed by a clicking sound resulting from the engagement of locking tabs 16 with respective locking holes 18, as described above.

As described above and shown in the drawings, each of first and second shell members 12, 14 is preferably made of a flexible material and is preferably semicircular in shape, with second shell member 14 having a smaller outer diameter than an inner diameter of first shell member 12 so that second shell member 14 is received by internal space 15 of first shell member 12 as shown in FIGS. 1, 4, 6 and 7. As shown in FIG. 8, first shell member 12 is symmetrical about central vertical line L1-L1 and symmetrical about central horizontal line L2-L2. As shown in FIG. 9, second shell member 14 is symmetrical about central vertical line X1-X1. By this construction, first and second shell members 12, 14 can be reversibly connected together without requiring any particular alignment of respective top/bottom ends of first and second shell members 12, 14.

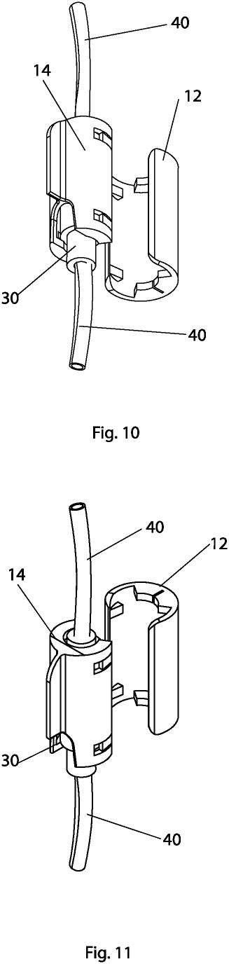

FIGS. 10-13 show tamper resistant device 10 engaging an access port 30 of a medical fluid line 40. FIGS. 10 and 11 are elevational perspective views with second shell member 14 engaging access port 30 and first shell member 12 disconnected from second shell member 12. FIG. 12 is an elevational side view showing tamper resistant device 10 in the assembled state engaging access port 30, and FIG. 13 is a cross-sectional view taken along line A-A in FIG. 12.

The access port 30 and corresponding fluid line 40 are first placed inside interior space 15 of second shell member 12 via open end 7 and then snapped into place, as shown in FIGS. 10 and 11. First shell member 12 can then be brought into engagement with second shell member 14 as described above with reference to FIGS. 1, 4, 5, 6 and 7 to thereby securely retain access port 30 within interior space 31 formed by first and second shell members 12, 14, as shown in FIGS. 12 and 13. As described above, cut away portion 24 of second shell member 14 is configured to accommodate, together with interior space 31 and opposite open ends 31a, 31b, access port 30 and corresponding medical fluid line 40, particularly during placement and removal of access port 30 and medical fluid line 40 relative to second shell member 14.

By the structural and positional arrangement of tamper resistant device 10, access port 30 and medical fluid line 40 shown in FIGS. 12 and 13, access port 30 cannot be tampered with by unauthorized persons without disconnecting first and second shell members 12, 14 from one another. While first and second shell members 12, 14 can be disconnected from one another (as shown in FIG. 2) with manual force and without requiring any tools as described above, tamper resistant device 10 effectively functions as a deterrent to prevent unauthorized persons from tampering with access port 30. That is, unauthorized persons would be discouraged from attempting to remove tamper resistant device 10, particularly in medical facilities, such as hospitals, where medical personnel are attending to and supervising patients. Furthermore, removal of tamper resistant device 10 without its proper replacement by unauthorized persons will serve as an indicator to alert authorized personnel that the access port 30 and medical fluid line 40 has been tampered with.

In the present embodiment, each of first shell member 12 and second shell member 14 has a unitary structure made of a single (one) piece of material. First and second shell members 12, 14 are preferably made of a suitable plastic material having some degree of flexibility to facilitate connection/disconnection between first and second shell members as described above. More specifically, the flexible nature of first shell member 12 permits one or both sides 13 to sufficiently flex outwardly to facilitate connection when second shell member 14 is pressed into interior space 15 of first shell member 12 and facilitate disconnection when second shell member 14 is removed/separated from interior space 15 of first shell member, as described above. As an example, the flexible plastic material selected for first and second shell members 12, 14 may be polypropylene. Alternatively, first and second shell members 12, 14 can be made of polyethylene, ABS (acrylonitrile butadiene styrene) or other suitable thermoplastic material. First and second shell members 12, 14 can be molded using injection molding technique, for example.

### Second Embodiment

FIGS. 14-28 show a second embodiment of the tamper resistant device, generally designated at 100, according to the present invention.

Tamper resistance device 100 has a shell member 110 and a locking element 115. Shell member 110 has a unitary structure made of a single (one) piece of material and is formed of a first portion 112 having a semi-annular cross-section and a second portion 114 having a semi-annular cross-section. First and second portions 112, 114 have a tubular construction, with second portion 114 having an outer diameter smaller than an outer diameter of the first portion. First portion 112 has an opening 113 providing access into an interior space 116 for accommodating and retaining therein a fluid access port and corresponding medical fluid line as further described below. Opposite ends of first portion 112 are provided with shoulder portions 120, 122 for aiding in retaining the fluid access port within interior space 116.

Second portion 114 extends from one end of first portion 112 and has smaller internal and external diameters than those of first portion 112. Second portion 114 has an opening 117 providing access into an interior space 118 for accommodating and holding the medical fluid line corresponding to the access port retained in the interior space 116 of first portion 112. Tamper resistance device 100 is also provided with opposite open ends 124, 126 corresponding to terminal end portions of first part 112 and second part 114, respectively. As further described below, open ends 124, 126 permit the passage therethrough of the fluid lines during use of tamper resistance device 100.

Referring to FIG. 20, opening 117 of second portion 114 has a tapered surface 128 that diverges from open end 126 towards interior space 116 of first portion 112. Tapered surface 128, together with openings 113, 117 and interior spaces 116, 118 of first and second portions 112, 114 facilitate placement and removal of the access port and corresponding fluid line relative to tamper resistance device 100.

First portion 112 is provided with a through-hole 119 proximate open end 124 for removably receiving locking element 115 as shown in FIGS. 15-17. Locking element 115 is configured to engage a portion the access port or the fluid line proximate the access port when the access portion and fluid line are retained within interior spaces 116, 118 of first and second parts 112, 114, as further described below with reference to FIGS. 23-28. The purpose of locking element 115 is to lock access port and corresponding fluid line in place to prevent tampering with the fluid access port by unauthorized persons. Authorized persons, such as medical professionals, would be provided with a suitable tool element (not shown) for removing locking element 115 to permit removal of the fluid access port and corresponding fluid line from tamper resistant device 100. Locking element 15 and through-hole 19 formed in first portion 112 therefore define means for removably holding/locking the access port and corresponding fluid line within interior spaces 116, 118 of first and second parts 112, 114.

Shell member 110 is preferably made of a suitable plastic material, such as polypropylene. Alternatively, shell member 110 can be made of polyethylene, ABS (acrylonitrile butadiene styrene) or other suitable thermoplastic material. Shell member 10 can be molded using injection molding technique, for example.

In an exemplary embodiment, locking element 115 can be in the form of a threaded element for threaded engagement with corresponding threads formed in through-hole 119. The tool element for removing locking element 115 may be in any suitable form, such as a screwdriver with a flat head or philips head, in which case the head of locking element 115 exposed to the exterior surface of first part 112 can be provided with a corresponding configuration to allow selective tightening and removal of locking element 115 using such tool element. Alternatively, the tool element may be in the form of a hex key (i.e., allen key or allen wrench) and the head of locking element 115 can be provided with a corresponding internal hexagonal recess. Locking element 115 may be made from a suitable plastic or metal material.

FIGS. 23-28 show how tamper resistant device 100 engages an access port 130 of a medical fluid line 140 to prevent tampering by unauthorized persons. Access port 130 may be, for example, a connection of medical fluid line 140 where one IV line connects into another (e.g., where a patient-controlled analgesia (PCA) IV line would connect into a maintenance IV fluid line.

With locking element 115 removed from through-hole 119 of first part 112, access port 130 and fluid lines 140 are accommodated in interior spaces 116, 118 of first and second parts 112, 114 as shown in FIGS. 23-28. This is accomplished by snapping first and second parts 112, 114 in place over access port 130, which is facilitated by opening 117 and open ends 124, 126 of corresponding first and second parts 112, 114, as well as by shoulder portions 120, 122 of first part 112 and tapered surface 128 of second part 114. Locking element 115 is then engaged with through-hole 119 using the locking tool as described above until it engages the portion of fluid line 140 proximate access port 130 to thereby lock access port 130 and fluid lines 140 in place relative to tamper resistant device 100. Removal of access port 130 and fluid lines 140 from tamper resistance device 100 can then be accomplished by removing locking element 15 (e.g., at least until locking element 115 disengages the portion of fluid line 140) using the tool element and manipulating access port 130 and fluid lines 140 relative interior spaces 116, 118, opening 117, and open end 120, 122 of corresponding first and second parts 112, 114 until they are removed from tamper resistance device 100.

By the structural and positional arrangement of tamper resistant device 100, access port 130 and medical fluid line 140 shown in FIGS. 23-28, access port 130 is effectively prevented from being tampered with by unauthorized persons without first disengaging locking element 115 from the portion fluid line 140 proximate access port 130. Since only authorized persons, such as medical personnel, have access to the tool element for removing locking element 115, tamper resistance device 100 effectively functions as a deterrent to prevent unauthorized persons from tampering with access port 130. That is, unauthorized persons would be discouraged from attempting to remove tamper resistant device 100, particularly because they do not have access to the tool element for removing locking element 115. Furthermore, should unauthorized persons have access to such tool element, the structural configuration of tamper resistant device 100 would discourage such persons from tampering with access port 130, particular in medical facilities such as hospitals, where medical personnel are attending to and supervising patients. Removal of or tampering with tamper resistant device 100 will serve as an indicator to alert authorized personnel that the particular access port 130 and medical fluid line 140 has been tampered with.

The tamper resistance device 100 has been described above to contain one locking hole 119 and one corresponding locking element 115. In an alternative embodiment, two are more locking holes and corresponding number of locking elements may be employed to further secure tamper resistance device 100 to access port 130 and corresponding fluid lines 140. In a further alternative embodiment, the locking hole and locking element may be omitted from tamper resistance device 100 since the snapping of first and second parts 112, 114 in place over access port 130 is configured to be sufficient to securely retain access port 130. While providing less security than when using a locking element engaged with a locking hole formed in shell member 110, the alternative embodiment without the locking element and hole is still sufficient to deter tampering with access port 130 by patients, since it will require two hands to remove shell member 110 to provide access to access port 130, as further described below.

### Third Embodiment

FIGS. 29-42 show another embodiment of the tamper resistant device, generally designated at 200, according to the present invention. Tamper resistant device 200 is configured for use as an end cap to secure any open port on a medical fluid line (e.g., IV line).

As shown in FIG. 42, tamper resistance device 200 has a cap member 210, a locking element 212, and disinfectant element 214. Cap member 210 has a tubular construction with a closed end 213 and an interior space 215 with an open end 216 and within which disinfectant element 214 is located. Space 215 is configured to receive an open port 230 of a medical fluid line 232 via open end 216 as shown in FIGS. 41 and 42. An inner surface of interior space 215 proximate closed end 213 is provided with a connecting structure 220 configured to receive and connect to a corresponding connecting structure of the open port on the medical fluid line. Disinfectant element 214 may comprise a cotton pad containing a disinfectant, such as isopropyl alcohol. When received within interior space 215 of cap member 210, open port 230 of medical fluid line 232 is brought into contact with disinfectant element 214 to place and maintain open port 230 in a disinfected state.

According to the present invention, a through-hole 218 is formed in a surface portion of cap member 210 proximate open end 216 so as to open into interior space 215. Through-hole 218 is configured to receive locking element 212 and lockingly engage open port 230 of medical fluid line 232 as shown in FIGS. 41 and 42. By this structural arrangement, open port 230 of medical fluid line 232 is maintained in a locked state relative to cap member 210 by locking element 212.

The purpose of locking element 212 is to lock open port 230 and corresponding medical fluid line 232 in place relative to cap member 210 to prevent tampering with the open port 230 by unauthorized persons. Authorized persons, such as medical professionals, would be provided with a suitable tool element (not shown) for removing locking element 212 to permit disconnection of open port 230 and corresponding medical fluid line 232 from tamper resistant device 200.

In an exemplary embodiment, locking element 212 can be in the form of a threaded element for threaded engagement with corresponding threads formed in through-hole 218. As described above with reference to the embodiment of FIGS. 14-28, a tool element for removing locking element 212 may be in any suitable form, such as a screwdriver with a flat head or Philips head, in which case the head of locking element 212 can be provided with a corresponding configuration to allow selective tightening and removal of locking element 212 using such tool element. Alternatively, the tool element may be in the form of a hex key (i.e., allen key or allen wrench) and the head of locking element 212 can be provided with a corresponding internal hexagonal recess.

Cap member 210 can be made from a plastic material, such as polypropylene. Alternatively, cap member 210 can be made of polyethylene, ABS (acrylonitrile butadiene styrene) or other suitable thermoplastic material. Cap member 210 can be molded using injection molding technique, for example. Locking element may be made from a suitable plastic or metal material.

By the structural and positional arrangement of tamper resistant device 200, open port 230 and medical fluid line 232 shown in FIGS. 41 and 42, open port 230 is effectively prevented from being tampered with by unauthorized persons without first disengaging locking element 212 using the tool member as described above. Since only authorized persons, such as medical personnel, have access to the tool element for removing locking element 212, tamper resistance device 200 effectively functions as a deterrent to prevent unauthorized persons from tampering with open port 230. That is, unauthorized persons would be discouraged from attempting to remove tamper resistant device 200, particularly because they do not have access to the tool element for removing locking element 212.

Furthermore, should unauthorized persons have access to such tool element, the structural configuration of tamper resistant device 200 would discourage such persons from tampering with open port 230, particularly in medical facilities such as hospitals, where medical personnel are attending to and supervising patients. Furthermore, removal of or tampering with tamper resistant device 200 will serve as an indicator to alert authorized personnel that the particular open port 130 and medical fluid line 232 has been tampered with.

From the foregoing construction and manner of use of tamper resistant devices 10, 100, 200 according to the present invention, it will be appreciated that each of tamper resistant devices 10, 100, 200 requires the use of two hands to facilitate its removal or disassembly relative to the access/open port and corresponding medical fluid lines. This effectively prevents unauthorized individuals, such as patients, that have peripherally inserted IV catheters, for example, from independently removing the tamper resistant device single-handedly. The locking element used in tamper resistant devices 100 and 200 as described above provides an additional, escalating level of tamper resistance which further prevents removal of these tamper resistance devices with even two hands due to the requirement for the locking tool to remove the locking element.

### Fourth Embodiment

A fourth embodiment of the tamper resistant device according to the present invention entails a modified version of any of the tamper resistant devices 10, 100 and 200 of the first, second and third embodiments. In this modified embodiment, the tamper resistant device 10, 100 and/or 200 further includes tamper detection means for detecting tampering with the tamper resistant device, thereby providing an additional layer of tamper resistance for both inpatient and outpatient settings. In one embodiment, the tamper detection means provides a visual indication of tampering and may a tamper detection device in the form of a sticker or any other tamper evident shrink band or wrap that is applied to a preselected region of the tamper resistance device. For example, the tamper detection device can be provided on a preselected overlapping region of connected first and second shell members 12, 14 of tamper resistant device 10, on preselected surface regions of shell members 112, 114 in contact with the access port/fluid line of tamper resistant device 100, and on a surface region of cap member 210 in contact with the open port/fluid line of tamper resistant device 200.

It will be appreciated that the tamper resistance devices described above with reference to foregoing embodiments can be made of a material other than plastic or thermoplastic materials. For example, the tamper resistance devices can be made using suitable metals, such as aluminum, copper, brass and combinations thereof.

As described above, the tamper resistant devices according to the present invention can effectively discourage and/or prevent unauthorized persons, such as patients, from tampering with access to in-situ intravenous (IV) medical catheter devices (placed either centrally or peripherally) and their corresponding medical fluid lines with access ports. They are particularly designed to prevent unauthorized patient access to the IV and to prevent any insertion/injection of non-prescribed medications/foreign substances through their IV access and associated ports.

The tamper resistant devices of the present invention are particularly well adapted for preventing unauthorized access of indwelling IV lines in a multitude of settings, such as hospitals, long-term facilities, skilled nursing facilities, in-patient rehabilitation units, and in the home health care setting. In light of today’s opioid crisis, the taper resistant devices according to the present invention are particularly effective in securing indwelling IV lines from tampering by individuals with opioid addictions, such as by having patients injecting narcotics and similar controlled substances into IV lines.

By securing and preventing the tampering of the IV and any access points, the tamper resistant devices of the present invention add additional depth to patient safety. Theses tamper resistant devices will help to minimize any potential blood stream infections risk, aid in detecting/identifying any potential abuse/self-injection of narcotic/opioid medications and in the prevention of sharing and/or selling of continuous or patient on-demand intravenously administered opioid medications with other patients (this practice is often referred to as “bumping”).

Thus, it will be appreciated that the tamper resistant devices of the present invention possess both visual and escalating physical deterrent properties for IV access ports, where no current visual or physical barrier currently exists in the inpatient or outpatient setting to prevent tampering.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

The foregoing is to be understood as being in every respect illustrative and exemplary, but not restrictive, and the scope of the invention disclosed herein is not to be determined from the detailed description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A tamper resistant device for holding a portion of an access port of a fluid line, the tamper resistant device comprising:
a first shell member having an interior space;
a second shell member having an interior space and configured to be removably connected to the first shell member, the second shell member having an outer diameter portions smaller than an inner diameter of the first shell member so that in a connected state, the second shell member is received by the interior space of the first shell member and an inner circumferential surface portion of the first shell member encircles and covers an outer circumferential surface portion of the second shell member, and in the connected state the first and second shell members together form an interior space for holding a portion of an access port of a fluid line to prevent tampering with the access port by unauthorized persons; and
means for removably connecting the first and second shell members together.

2. The tamper resistant device of claim 1, wherein the means for removably connecting the first and second shell members together comprises at least one locking element formed in the interior space of the first shell member and at least one locking hole formed in the second shell member for removably receiving the at least one locking element.

3. The tamper resistant device of claim 2, wherein the at least one locking element comprises a plurality of locking elements, and the at least one locking hole comprises a plurality of locking holes for removably receiving the respective plurality of locking element.

4. The tamper resistant device of claim 1, wherein the first and second shell members are made of a flexible material.

5. The tamper resistant device of claim 1, wherein each of the first shell member and the second shell member has a unitary structure made of a single piece of flexible material.

6. The tamper resistant device of claim 1, wherein each of the first shell member and the second shell member has a generally semi-circular cross-section.

7. The tamper resistant device of claim 1, further comprising tamper detection means for providing a visual indication of tampering with the first and/or second shell members.

8. The tamper resistant device of claim 7, wherein the tamper detection means comprises one of a tamper detection sticker and tamper evident shrink band or wrap applied to a preselected region of the first and/or second shell members.

9. A tamper resistant device for holding a portion of an access port of a fluid line, the tamper resistant device comprising:
a shell member having a unitary structure made of a single piece of material, the shell member having a first tubular portion having a first interior space and a second tubular portion extending from the first tubular portion and having a second interior space, the first and second interior spaces being configured to receive and retain therein an access port of a fluid line to prevent tampering with the access port by unauthorized persons; and
means for removably holding and retaining the access port within the first and second interior spaces of the first and second tubular portions of the shell member.

10. The tamper resistant device of claim 9, wherein the means for removably holding and retaining the access port comprises a locking element separate from the shell member and a through-hole formed in the first tubular portion of the shell member for removably receiving the locking element so as to engage the access port retained in the first interior space of the first tubular portion.

11. The tamper resistant device of claim 10, further comprising a tool member for removing the locking element from the through-hole of the first tubular portion to permit removal of the access port from the first and second tubular portions.

12. The tamper resistant device of claim 9, wherein the second tubular portion has an outer diameter smaller than an outer diameter of the first tubular portion.

13. The tamper resistant device of claim 9, further comprising tamper detection means for providing a visual indication of tampering with the shell member.

14. The tamper resistant device of claim 13, wherein the tamper detection means comprises one of a tamper detection sticker and tamper evident shrink band or wrap applied to a preselected region of the shell member.

15. A tamper resistant device for closing an open port of a fluid line, the tamper resistant device comprising:
a cap member having an open end providing access into an interior space of the cap member configured to receive and removably retain therein an open port of a fluid line; and
means for removably holding and retaining the open port within the interior space of the cap member to prevent unauthorized tampering with the open port by unauthorized persons.

16. The tamper resistant device of claim 15, wherein the means for removably holding and retaining the open port comprises a locking element separate from the cap member and a through-hole formed in the cap member for removably receiving the locking element so as to engage and retain the access port within the interior space of the cap member.

17. The tamper resistant device of claim 15, further comprising a disinfectant element disposed within the interior space of the cap member for contacting and maintaining in a disinfected state the access port disposed in the interior space.

18. The tamper resistant device of claim 15, further comprising tamper detection means for providing a visual indication of tampering with the cap member.

19. The tamper resistant device of claim 18, wherein the tamper detection means comprises one of a tamper detection sticker and tamper evident shrink band or wrap applied to a preselected region of the cap member.

20. The tamper resistant device of claim 15, wherein the cap member has a closed end opposite the open end.
